(19) United States
(12) Patent Application Publication
(10) Pub. No.: US 2006/0135855 A1
(43) Pub. Date: Jun. 22, 2006

(54) Method for determining normal measurements for a patient

(75) Inventors: Yasser H Alsafadi, Yorktown Heights, NY (US); Larry J Eshelman, Ossining, NY (US); Xinxin Zhu, Croton-On-Hudson, NY (US); Amr F Yassin, Ossining, NY (US); Doug Sluis, W Mukilteo, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 10/537,874

(22) PCT Filed: Dec. 3, 2003
(86) PCT No.: PCT/IB03/05801

§ 371 Date: Jun. 7, 2005

Related U.S. Application Data
(60) Provisional application No. 60/435,235, filed on Dec. 20, 2002.

(51) Int. Cl. A61B 5/00

(52) U.S. Cl. 600/300

(57) ABSTRACT

Method and system for determining a norm to apply to a patient being examined in which at least one physical characteristic of the patient is determined from the examination and guidelines relating thereto are obtained. Information about the patient other than from the examination and established norms for each characteristic are also obtained. An established norm to be applied to the patient is selected based on the determination of the characteristic(s), the guidelines relating to the determination thereof, and the information about the patient other than from the examination. The selection of the norm is performed by an inference engine. The norm is applied to the characteristic, e.g., a measured anatomical feature, to obtain a normal value therefor. The measured anatomical feature is compared to the normal value to determine whether it is indicative of a normal or abnormal condition.

The present invention relates generally to a method and system for determining a normal value or range of normal values of a physical characteristic of a patient and more specifically to a method and system for determining a norm to be used for assessing normalcy of an anatomical feature.

It is important to know whether fetuses are growing normally for the purpose of detecting potential abnormal growth conditions, such as intrauterine growth retardation, in order to enable treatment of such conditions, when possible. When determining whether a fetus is growing normally, current medical modalities are used to obtain physical measurements of the fetus. Values relating to the fetus's growth are derived from the measurements using known formula and a determination is then made whether the derived values fall within a range of values corresponding to those of a normal condition for the fetus, i.e., a fetus undergoing normal growth.

The range of values corresponding to a normal condition varies for each fetus because they depend on numerous criteria. For example, some criteria include the geographic region in which the mother lives, the ethnic background of the mother and the mother and fetus's health history.

To date, there is no easy way to obtain the range of values for every individual. Typically though, norms are used. Norms are an accepted metric or criteria for a specific clinical evaluation, one example being a growth chart. Typically, norms are stored in medical modalities used in patient examination, such as in ultrasound imaging equipment. Norms usually take the form of a table or a formula.

There are numerous norms which correlate measurements of a physical characteristic of a fetus to the gestational age for the purpose of determining whether the fetus is growing at a normal rate. The measurements correlated to gestational age may be any measurable parameter relating to the physiology of the fetus, such as the length or size of a particular bone, the diameter of a particular bone, the size of a particular organ, the circumference of a part of the fetus (head or abdomen) and other similar characteristics or ratios of two or more of the measurements. These measurements are usually obtained during an ultrasound procedure but may be obtained in other ways known to those skilled in the art.

Once a measurement of a particular bone or organ or part of the fetus is obtained, reference is made to a norm, i.e., a table or formula, to obtain a gestational age corresponding to that measurement In other words, the norm expresses what the age of a fetus undergoing normal growth should be when it has that measurement. Each table or formula also typically includes an error or standard deviation to thereby define range of the gestational age. If the gestational age of the fetus, as determined from an assumed date of conception, is outside of the gestational age range obtained from the table or formula, then this would be indicative of abnormal growth. On the other hand, when the gestational age of the fetus as determined from the assumed date of conception falls within the gestational age range obtained from the table or formula, then normal growth is indicated.

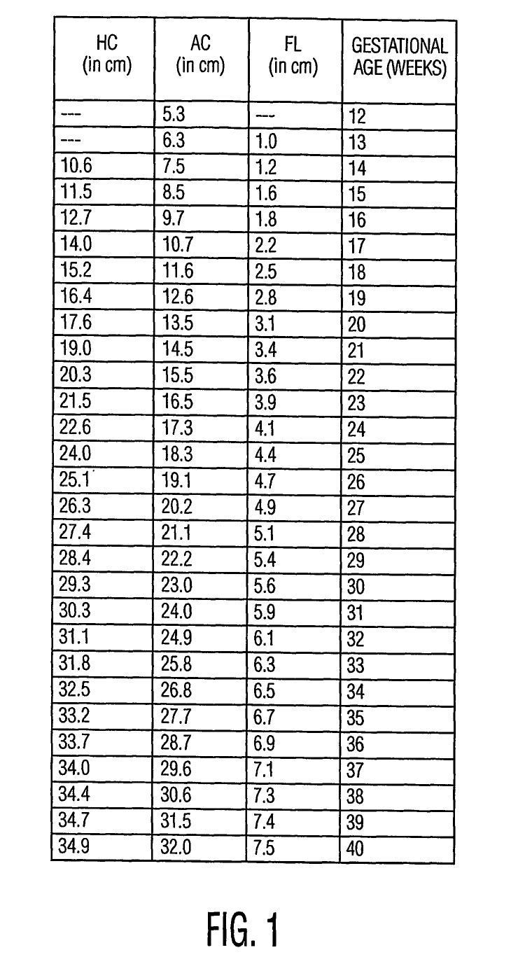

FIG. 1 shows an example of a table relating several characteristics of a fetus to gestational age, namely, the circumference of the head (designated HC), the circumference of the abdomen (designated AC) and the length of the femur (designated FL). The data in this table is derived by Hansmann, Hackeloer, Staudach and Wittman and is set forth in “Ultrasound Diagnosis in Obstetrics and Gynecology”, Springer-Verlag, New York 1986. The table provides an expectation of the gestational age for measurements of the head circumference, abdomen circumference and femur length within measurement ranges, e.g., a measurement range of the length of the femur is from 1.0 cm to 7.5 cm. Thus, in use for example, if the femur length of a fetus is measured as 3.1 cm, the expected gestational age would be 20 weeks. If the age of the fetus based on the assumed date of conception is for example 30 weeks, this would likely be an indication of abnormal growth.

Instead of a table, a formula can be used to obtain an expected gestational age range. One formula derived by Hadlock, Deter, Harrist and Park and set forth in “Estimating Fetal Age: Computer Assisted Analysis of Multiple Fetal Growth Parameters”, Radiology 1984, 152: 497-501, is:
10.35+(2.460*FL)+(0.170*(FL²))=Gestational Age (weeks)

wherein FL is measured femur length expressed in cm. This formula is applicable for a range of femur length from 0.64 cm to 8.0 cm. The standard deviation, which provides a range of the gestational age, varies from 1.38 weeks to 3.12 weeks as follows:

| | | Standard |
|---|---|---|
| Lower limit (weeks) | Higher limit (weeks) | deviation |
| 12 | 18 | 1.38 |
| 18 | 24 | 1.80 |
| 24 | 30 | 2.08 |
| 30 | 36 | 2.96 |
| 36 | 42 | 3.12 |

Another formula derived by Hohler and Quetel and set forth in “Fetal Femur Length: Equations for Computer Calculation of Gestational Age from Ultrasound Measurements”, American Journal of Obstetrics and Gynecology, Vol. 143, No. 4, Jun. 15, 1982, pp.479-481 is:
9.18+2.67*FL+0.16*(FL²)=Gestational Age (weeks)
wherein FL is measured femur length expressed in cm. This formula is applicable for a range of femur length from 1.0 cm to 8.0 cm.

In a similar manner, tables and formulas are derived for other measurable physical characteristics, such as abdominal circumference (AC), biaparietal diameter (BPD), head circumference (HC), occipital-frontal diameter (OPD) and others as set forth in DICOM (Digital Imaging and Communications in Medicine) Supplement 26.

A problem arises in that each table or formula is likely to provide a different range of expected gestational age so that when one table or formula is used, the measured physical characteristic might be indicated as normal growth but if another table or formula were to be used, the same measured physical characteristic would be indicative of abnormal growth.

Although gestational age is used above as an indicator of normal or abnormal growth. Estimated fetal weight (EFW) can also be used. There are norms which correlate a measured physical condition, such as the length of a bone or circumference of the head or abdomen, to estimated fetal weight. In this case, if the estimated fetal weight determined from the assumed date of conception (obtained from a table for example) deviates from the estimated fetal weight derived from the norm, then this would be indicative of an abnormal condition.

It is also important to know whether organs and bones are in a normal proportion to the rest of the body and growing at a normal rate because problems may arise if a particular organ is not in the proper proportion with the rest of the body. For example, the heart and the heart valves usually grow commensurate with age and if a heart valve is not growing at the proper rate or is otherwise not in proportion to the rest of the body, it may be indicative of cardiological problems.

Accordingly, it is an object of the present invention to provide a method and system for determining a range of normal measurements of a physical characteristic of a patient. Herein, a “patient” is used to refer to an object or organism that grows such as a fetus as well as children and adults. A “physical characteristic” may be any property (length, diameter, etc.) of an anatomical feature (bone, organ, etc.).

It is another object of the present invention to provide a method for “patient centered” care, i.e., determining the particular needs of individual patients and then tailoring care to them.

It is another object of the present invention to provide a method and system for determining whether measurements of a fetus are within a range of normal measurements individually selected for that patient.

It is another object of the present invention to provide a method and system for determining whether measurements of an anatomical feature within a range of normal measurements individually selected for that patient.

In order to achieve these objects and others, a method for determining a norm to apply to a patient in accordance with the invention comprises examining the patient, determining or measuring at least one physical characteristic of the patient from the examination, obtaining guidelines relating to the determination or measurement of each characteristic from the examination, obtaining information about the patient other than from the examination, obtaining established norms for each characteristic and selecting one of the established norms to be applied to the patient based on the determination or measurement of the characteristic, the guidelines relating to the determination thereof, and the information about the patient other than from the examination. The selection of the norm, i.e., the determination of the most appropriate norm for the patient, may be performed by an inference engine.

If the patient is a fetus and the norm is one which provides as output an expected gestational age or age range upon input of the physical characteristic, then the characteristic as determined from the examination is input into the norm to obtain the expected gestational age or age range. The age of the fetus (as determined for example based on the assumed date of conception) is then compared to the expected age or age range to see whether it is proximate the expected age or within the expected range (and thus indicative of a normal growth condition). Otherwise, if the age of the fetus determined from the assumed date of conception is relatively distant from the expected age or outside of the expected age range, e.g., higher, then this would be indicative of abnormal growth.

Depending on the norm, the norm might be applicable to a physical characteristic which is derived from a plurality of measured anatomical features, e.g., a ratio of two anatomical features (such as a ratio of the head circumference to abdomen circumference (HC/AC) or femur length to abdomen circumference (FL/AC)). In this case, a normal value or a range of normal values for the physical characteristic, i.e., the ratio, is obtained and compared to a ratio derived from the measured anatomical feature of the fetus to determine whether the ratio of anatomical features is indicative of a normal or abnormal condition.

The examination may be performed using any medical modality such as an ultrasound imaging system, an MRI (magnetic resonance imaging) system, a CT system and other imaging modalities. The anatomical feature may be a length, size or diameter of a bone in the patient or a circumference of a head or abdomen.

In another embodiment, a norm for use for a patient is determined by examining the patient, determining or measuring at least one physical characteristic of the patient from the examination and expressing it in XML syntax. Further, guidelines relating to the determination of the characteristic are obtained and described using a methods ontology based on semantic web technology. Information about the patient other than from the examination is obtained from a hospital or departmental information system using HL7 messaging which uses XML syntax. Established norms for each characteristic, e.g., an anatomical feature or a ratio of two anatomical features, are obtained such as from one or more libraries thereof or from memory in the medical modality, and expressed in XML syntax. Thereafter, an inference engine is provided to receive the determination or measurements of each characteristic and the guidelines relating thereto and the information about the patient other than from the examination and selects one of the established norms to be applied to the patient based thereon.

A system for determining a norm to be used for a patient in accordance with the invention comprises a modality for obtaining measurements of a physical condition of a patient and enabling characteristics relating to the physical characteristics of the patient to be derived, a processor coupled to the modality, a repository of medical information about the patient coupled to the processor, at least one library of norms relating to the physical characteristics of the patient coupled to the processor, and at least one library coupled to the processor and containing clinical guidelines associated with a procedure used by the modality to derive the measurements. The processor receives the derived characteristics of the patient from the modality, the measurements from the modality, medical information about the patient from the repository of medical information, the norms from each library of norms and the clinical guidelines from each library of clinical guidelines and selects one of the norms which is most appropriate for use with the patient. Data on the selected norm is provided, including the upper and lower limits on the range of the input parameter and various details about the origins of the norm. By applying the norm to the measurements of the patient from the modality, the normalcy or abnormalcy of the patient can be deduced.

In one embodiment, the links between the processor, the hospital or departmental information system, each library of norms and each library of clinical guidelines are Internet or Intranet connections.

To facilitate easy communications, the modality is arranged to express the measurements of the patient and the derived values relating to physical characteristics of the patient in XML syntax, and the repository of medical information about the patient is arranged to remotely communicate with the processor using HL7 messaging using XML syntax. Also, each library of norms is arranged to express the norms in XML syntax and each library of guidelines is arranged to describe the guidelines using a methods ontology based on semantic web technology.

The invention, together with further objects and advantages hereof, may best be understood by reference to the following description taken in conjunction with the accompanying drawings, wherein like reference numerals identify like elements and wherein:

FIG. 1 is a table showing derived correlations between anatomical features and gestational age.

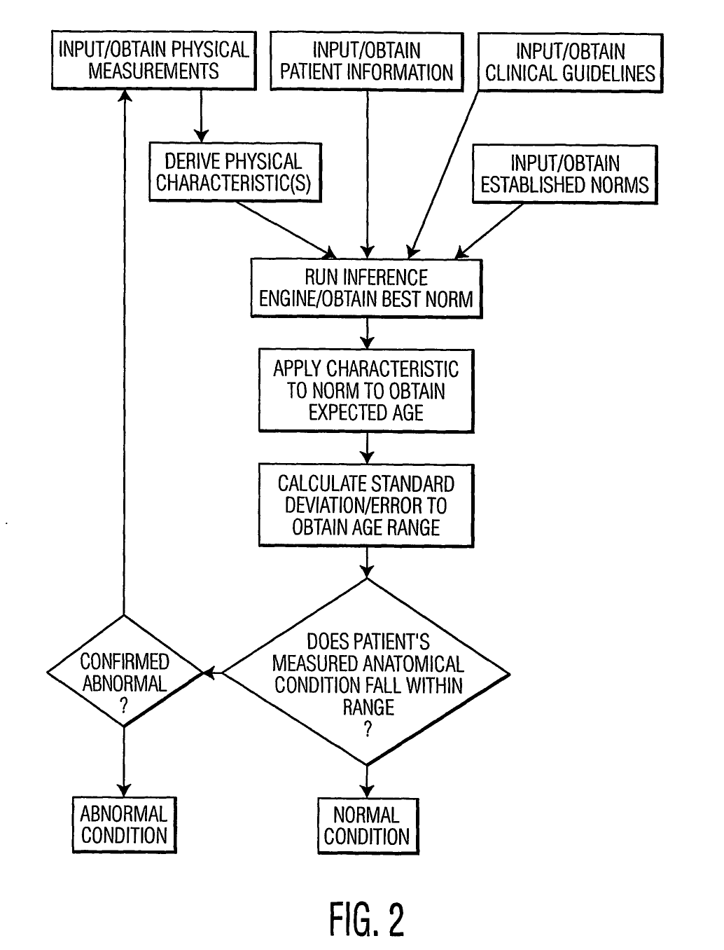

FIG. 2 is a flowchart showing the method in accordance with the invention.

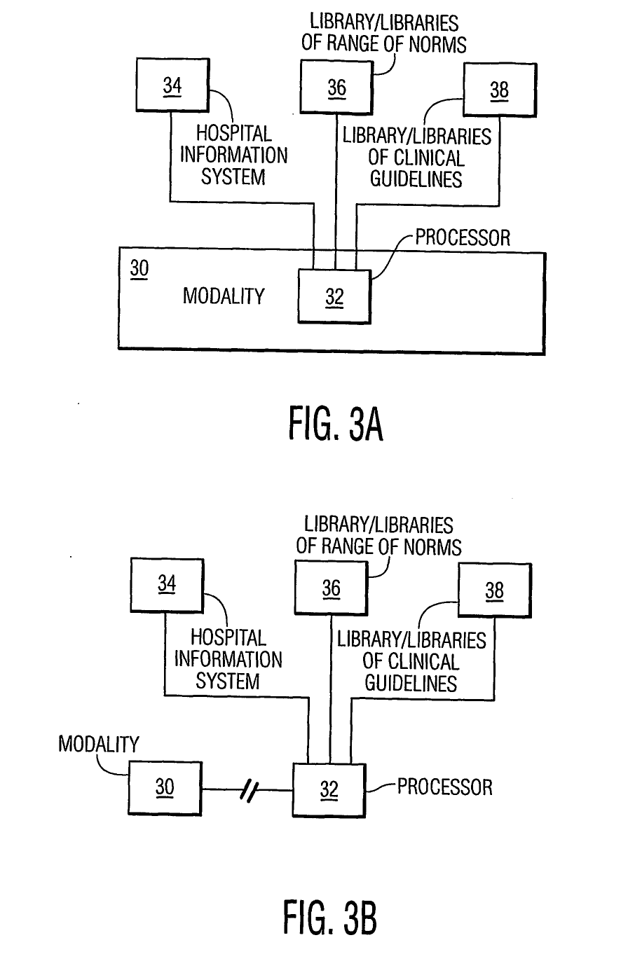

FIGS. 3A and 3B are schematic flowcharts showing the manner in which the present invention functions.

Referring initially to FIG. 2, a method in which a recommendation on the normalcy of one or more physical characteristics of a fetus is described.

One or more physical measurements of the fetus are obtained during an examination of the mother at Step 10. This may entail measuring anatomical features of the fetus, for example, using an ultrasound system during a scanning procedure with the measurements being determined during the scanning or with the measurements being determined off-line at a workstation. The measurements of the fetus may include the femur length (FL), head circumference (HC), abdomen circumference (AC) and biaparietal diameter (BPD) as well as other anatomical features as listed in DICOM Supplement 26. For further processing and use in the invention, the measurements are preferably expressed in XML (extensible mark-up language) format.

Physical characteristics are derived from the measurements at Step 12. A physical characteristic may either be the measurement itself or a value derived from one or more measurements, such as a ratio of two measurements. The measurement or value is selected based on available norms, i.e., it must be one for which a norm is known.

Information about the fetus, other than the measurements of the physical characteristics obtained during the examination, is also obtained at Step 14 from one or more hospital information systems, and possibly departmental information systems. The hospital information system may be queried to provide the information by a query containing the patient's identification and the requested information. The requested information may include the fetus's or mother's age, weight, gender and ethnicity.

To communicate with the hospital information system, the system which determines which norm to apply is resident on a processor (as described below with reference to FIGS. 3A and 3B) and uses a form of messaging conducive to information exchange such as HL7 messaging. This type of messaging also preferably uses XML syntax for further processing of the information from the hospital information system. A vocabulary is provided, either from the hospital information system or on the processor, and used to indicate and define any coded terms in the information provided by the hospital information system. Information about the mother can also be provided, such as information about the uterus and the presence of other fetuses.

Clinical guidelines associated with the procedure used to derive the physical characteristics are retrieved from one or more libraries of such clinical guidelines at Step 16 and preferably described using a methods ontology based on semantic web technology. An ontology (knowledge base) is a conceptualization of a domain of guidelines. One skilled in the art would understand the manner in which a methods ontology based on semantic web technology is configured and used.

The clinical guidelines provide the steps associated with the procedure and highlight the measurements required and any additional steps/measurements to be taken to verify if a ratio is abnormal.

Established norms are retrieved at Step 18 from one or more libraries of norms and preferably expressed in XML syntax. The norms may be resident on software or hardware of the medical apparatus used during the examination of the fetus, or may be accessed through an Internet or Intranet connection by the processor.

The measurements obtained during the examination of the fetus, the clinical guidelines used to derive the physical characteristics, the information about the fetus (and optionally mother) and the established norms are input to an inference engine resident on the processor at Step 20. The inference engine derives a norms recommendation based on the input data, i.e., the most appropriate norm for use with the input data. The norms recommendation is a selected norm, i.e., either a table or formula, to which the physical characteristic obtained or derived from the examination will be applied to obtain an indication of normalcy of the physical characteristic with respect to gestational age.

For example, the selected norm may be the formula derived by Hadlock et al. set forth above (10.35+(2.460*FL)+(0.170*(FL²))=Gestational Age (weeks) wherein FL is measured femur length expressed in cm). The norm would also have associated limits of the range of input values, i.e., a range of permitted input measurements, in this case from 0.64 cm to 8.0 cm) and would include its origin. The origin would specify the code value of the DCMR or local coded concept, the coding scheme designator of the DCMR or local coded concept and the code meaning of the DCMR or local coded concept.

The measurement is then input to the selected norm to provide the expected gestational age or age range (Step 22).

A standard deviation or error is determined, if any is associated with the norm, in order to provide a range of the gestational age (Step 24). That is, if the femur length was measured as 2.0 cm, the gestational age provided by the formula would be 15.95 weeks with the deviation of 1.38 weeks so the gestational age range would be 14.57 weeks to 17.33 weeks.

The age of the fetus obtained from the assumed date of conception would be compared to this range for the purpose of assessing growth normalcy or abnormalcy (Step 26). Thus, if the age from the assumed date of conception is 25 weeks, it would be outside of the range of normal growth and thus indicative of abnormal growth.

In the event an abnormal condition is determined, then a determination is made whether the determination of the abnormal condition is the first determination or a subsequent confirmation of the first determination (Step 28). If it is the first determination of an abnormal condition, then a feedback loop to the input or obtaining of the physical measurements (Step 10) may be used to confirm the abnormal condition. That is, by providing a feedback and requiring the re-entry or re-obtaining of the physical measurements, errors in data entry or measurement errors can be reduced. Also, additional measurements may be obtained after an abnormal condition is initially determined to aid in the confirmation of the abnormal condition in a subsequent analysis of the physical measurements. Once the abnormal condition is confirmed, then the diagnosis results in an abnormal condition.

Typically, the selected norm includes not only the value of the 50^{th} percentile but can and should also include information that describes the distribution width, e.g., the standard deviation. Clinically, physicians want to know if the biometric value is outside some range as the 10/90 (below the 10^{th} percentile or above the 90^{th} percentile) or 5/95 or 2 STD (standard deviations 3/97) range.

Referring now to FIGS. 3A and 3B, the components used in the method in accordance with the invention are set forth in a flow chart. The modality 30, e.g., an ultrasound machine, is present in an examination room and the results of the examination are obtained and analyzed to derive one or more measurements of anatomical features of the fetus by an ultrasound technician operating the ultrasound machine. The measurements or values derived from the measurements are preferably expressed in XML syntax.

The measurements are provided to a processor 32 which may be integral with or remote from the ultrasound machine.

The processor 32 is also coupled to a hospital information system (HIS) 34 or other repository of medical information about the fetus and optionally the mother. The hospital information system 34 provides data about the fetus and mother upon request by the processor 32. The specific type of information to be obtained from the hospital information system 34, e.g., age, weight, ethnicity, gender, is determined by the processor 32.

Instead of general hospital information, departmental information systems can be used to provide more specific information, for example, a radiology information system (RIS) which would provided radiological information, a cardiological information system (CIS) which would provide cardiological information, etc.

The processor 32 is also coupled to one or more libraries of norms 36 which contain data about each norm including for example, when the norm is an equation, the author, the application, the file type, the source, the input parameter and range, the output parameter and unit and an equation for determining the output parameter from the input parameter. Each norm can be modeled to include four aspects, a target concept which is the concept to be computed using the norm, a list of dependencies which is a list of concepts on which the computation of the target concept depends, an equation which is a function that provides the target concept based on the dependency and one or more applicability constraints. With respect to the applicability constraints, some norms are applicable in certain contexts and not others. These constraints are the criteria which determine whether one norm is better than another.

A norm can also be a table listing different values of the physical measurement and an output parameter and unit for each different value. The table would also include the author, the file type, the source and any applicability constraints. The table could also include a standard deviation.

The processor 32 is also coupled to one or more libraries 38 containing clinical guidelines associated with the procedure that obtained the measurements or the derivation of the physical characteristics.

The hospital information system 34, the libraries of norms 36 and the libraries of clinical guidelines 38 may be operated independently of one another, each as a stand alone system. Each of them is however linked to the processor.

Once the processor 32 receives all of the inputs, it derives a norm recommendation, i.e., selects one of the norms from the library 34 based on the input data. This norm would be used to analyze the physical characteristics obtained or derived from the examination. The processor 32 displays the norm recommendation with any or all of its associated information, e.g., its derivation, origins, author, limitations. The norm recommendation may be entered into a report or workstation at which time, the measured anatomical feature of the patient, or a derived value/ratio based on one or more measured anatomical features would be entered into the norm for the purpose of assessing whether the physical characteristic of the patient (the measured anatomical feature or ratio of anatomical features) is proximate the output of the norm or within a range provided by the norm. If so, the patient would be diagnosed with normal growth. On the other hand, a determined physical characteristic distant from the output of the norm or outside of the range provided by the norm could be considered abnormal growth.

As shown in FIG. 3A, the processor 32 is resident in the modality 30 or otherwise arranged in connection therewith. In this case, the modality 30 would be constructed to provide the functions of the processor 32, i.e., include appropriate communications software and hardware to enable connections to the hospital information system 34, the libraries of norms 36 and the libraries of clinical guidelines 38 to be established.

In the alternative, the modality 30 could be constructed independent of the processor 32 which performs the functions in accordance with the invention and thus the processor 32 could be implemented as a service for independently operating modalities. Thus, as shown in FIG. 3B, the links between the processor 32 and the hospital information system 34, the libraries of norms 36 and the libraries of clinical guidelines 38 may be connections over the Internet or Intranet connections. The processor 32 could also be wired directly to the hospital information system 34, the libraries of norms 36 and the libraries of clinical guidelines 38. In these embodiments, the processor 32 would be independent of the modality 30.

Although reference is made above to determining a norm of a physical characteristic of a fetus, the method and system in accordance with the invention are applicable to any growing objects having quantifiable growth charts or curves including people, children in particular, as well as an organ or bone which is expected to grow in proportion to the rest of the body.

That is, the present invention is applicable is determining whether organs and bones of children and adults are in normal proportions. For example, in accordance with the invention, it is possible to determine whether heart valves are at a normal size in order to detect myocardial infractions which might arise if the heart valves were not in a proper proportion. In this case, the size of the heart valves are measured, e.g., using ultrasound, and additional information about the patient is obtained in the manner described above. Clinical guidelines about the heart valve measurement procedure are also obtained. This information is processed to obtain a value or range of values indicative of a heart valve at a normal size for the individual. Standard deviations could also be obtained. The measured size of the heart valve would be compared to the obtained value or range of values to assess whether the heart valve is a normal size or not. In the same manner, the invention can be used to determine the normalcy of bones and other organs (i.e., whether they are in a normal proportion) as well as the normal or abnormal growth of organs and bones.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. A method for determining a norm for use in assessing normalcy of a patient being examined, comprising the steps of:
determining at least one physical characteristic of the patient from the examination;
obtaining guidelines relating to the determination of the at least one characteristic from the examination;
obtaining information about the patient other than from the examination;
obtaining established norms for the at least one characteristic; and
selecting one of the established norms to be applied to the patient based on the determination of the at least one characteristic, the guidelines relating to the determination of the at least one characteristic and the information about the patient other than from the examination.

2. The method of claim 1, wherein the at least one characteristic is an anatomical feature, further comprising applying the norm to the anatomical feature to obtain a normal value for a measurable anatomical feature or a range of normal values for a measurable anatomical feature and comparing the determined anatomical feature of the patient to the normal value or range of normal values to determine whether the determined anatomical feature is indicative of normalcy of the patient or abnormalcy of the patient.

3. The method of claim 1, wherein the at least one characteristic is a ratio of two anatomical features, further comprising applying the norm to the ratio to obtain a normal value for the ratio or a range of normal values for the ratio and comparing a ratio derived from the determined anatomical features of the patient to the derived normal value or range or range of normal values to determine whether the at least one anatomical feature falls is indicative of normalcy of the patient or abnormalcy of the patient.

4. The method of claim 1, wherein the step of performing the examination comprises the step of performing an ultrasound examination of the patient.

5. The method of claim 4, wherein the step of determining the at least one characteristic comprises the step of measuring a length, size or diameter of a bone in the patient or measuring a circumference of a head or abdomen.

6. The method of claim 4, wherein the step of determining the at least one characteristic comprises the steps of measuring at least two anatomical features and calculating a ratio of two anatomical features, guidelines relating to the ratio of the two anatomical features being obtained.

7. The method of claim 1, further comprising expressing the determination of the at least one characteristic in XML syntax.

8. The method of claim 1, further comprising expressing the established normal ranges of the at least one characteristic in XML syntax.

9. The method of claim 1, wherein the information about the patient other than obtained from the results of the examination is obtained from a hospital or departmental information system using HL7 messaging which uses XML syntax.

10. The method of claim 9, wherein the information about the patient is coded in the hospital or departmental information system, further comprising accessing a vocabulary to decode the information about the patient prior to the selection of one of the established norms.

11. The method of claim 1, wherein the selection of one of the established norms is performed by an inference engine.

12. A method for determining a norm for use in assessing normalcy of a patient being examined, comprising the steps of:
determining at least one physical characteristic of the patient from the examination;
expressing the determination of the at least one characteristic in XML syntax;
obtaining guidelines relating to the determination of the at least one characteristic from the examination, the guidelines being described using a methods ontology based on semantic web technology;
obtaining information about the patient other than from the examination from a hospital or departmental information system using HL7 messaging which uses XML syntax;
obtaining established norms for the at least one characteristic;
expressing the established normal ranges of the at least one characteristic in XML syntax; and
providing an inference engine to receive the determination of the at least one characteristic, the guidelines relating to the determination of the at least one characteristic and the information about the patient other than from the examination and selecting one of the established norms to be applied to the patient based thereon.

13. A system for determining a norm for use in assessing normalcy of a patient, comprising:
a modality for obtaining physical measurements of a patient and enabling at least physical characteristic of the patient to be derived;
a processor coupled to said modality;
a repository of medical information about the patient coupled to said processor,
at least one library of norms relating to characteristics of the patient coupled to said processor; and
at least one library coupled to said processor and containing clinical guidelines associated with a procedure used by said modality to derive the characteristics; said processor being arranged to receive the characteristics of the patient derived by said modality, medical information about the patient from said repository of medical information, the norms from said at least one library of norms and the clinical guidelines from said at least one library of clinical guidelines and select one of said norms which is most appropriate for use with the patient.

14. The system of claim 13, wherein links between said processor, said repository of medical information, said at least one library of norms and said at least one library of clinical guidelines are Internet or Intranet connections.

15. The system of claim 13, wherein said modality is arranged to express the measurements of the patient and the derived values relating to physical conditions of the patient in XML syntax.

16. The system of claim 13, wherein said repository of medical information about the patient is arranged to remotely communicate with said processor using HL7 messaging using XML syntax.

17. The system of claim 13, wherein said at least one library of norms is arranged to express the norms in XML syntax.

18. The system of claim 13, wherein said at least one library of guidelines is arranged to describe the guidelines using a methods ontology based on semantic web technology.

19. The system of claim 13, wherein said processor is arranged to apply the norm to the measurements obtained by said modality to provide an indication of a normal or abnormal condition.

20. The system of claim 13, wherein said processor is arranged in connection with said modality.
